(19) United States
(12) Patent Application Publication
(10) Pub. No.: US 2025/0171742 A1
(43) Pub. Date: May 29, 2025

(54) BIOREACTOR FOR CELL EXPANSION AND CULTIVATION

(71) Applicant: UNIVERSITY OF GEORGIA RESEARCH FOUNDATION, INC., Athens, GA (US)

(72) Inventors: Daniel Christian Shah, Athens, GA (US); Cheryl Teresa Gomillion, Athens, GA (US); Morgan Elizabeth Mantay, Athens, GA (US)

(21) Appl. No.: 18/841,968

(22) PCT Filed: Feb. 28, 2023
(86) PCT No.: PCT/US2023/014078

§ 371 Date: Aug. 27, 2024

Related U.S. Application Data
(60) Provisional application No. 63/314,771, filed on Feb. 28, 2022.

(51) Int. Cl. C12N 5/0775; C12M 1/00; C12M 1/12; C12M 3/00

(52) U.S. Cl. CPC C12N 5/0663; C12M 23/48; C12M 25/04; C12M 29/18

(57) ABSTRACT

A bioreactor includes a vessel comprising a wall with an interior surface defining a volume, and a fluid outlet: a cell support insert removably disposed within the vessel: a lid constructed to close the vessel; a fluid inlet; and a fluid distributor in fluid communication with and arranged to receive a fluid flow from the fluid inlet. The fluid distributor has a plurality of fluid delivery ports constructed to deliver fluid onto the cell support insert. The fluid distributor is arranged to apply a shear force of 0.005 Pa to 0.15 Pa to the cells when cell culture media is circulated at a rate of 25 mL/min to 125 mL/min.

### CONTINUING APPLICATION DATA

This application claims the benefit of U.S. Provisional Application Ser. No. 63/314,771, filed Feb. 28, 2022, which is incorporated by reference herein.

### GOVERNMENT FUNDING

This invention was made with government support under grant number 1648035 awarded by the National Science Foundation. The government has certain rights in the invention.

### FIELD

The present disclosure relates to bioreactors and bioreactor systems for the expansion and cultivation of cells, such as stem cells. The present disclosure further relates to bioreactors that can support dynamic cell culture conditions for 2D and 3D cell cultures.

### BACKGROUND

Mesenchymal stromal cells (MSCs) have been widely studied for biomedical research and clinical applications, yet the extent of their application is still being investigated. In addition to MSCs, the products they secrete (proteins, exosomes, etc.) are also widely beneficial for therapeutic application. Improved systems and methods for reliable and efficient production of MSCs are desired.

### SUMMARY

The present disclosure relates to bioreactors and bioreactor systems for the expansion and cultivation of cells, such as stem cells. According to an embodiment, a bioreactor includes a vessel comprising a wall with an interior surface defining a volume, and a fluid outlet; a cell support insert removably disposed within the vessel; a lid constructed to close the vessel; a fluid inlet; and a fluid distributor in fluid communication with and arranged to receive a fluid flow from the fluid inlet. The fluid distributor has a plurality of fluid delivery ports constructed to deliver fluid onto the cell support insert. The fluid inlet may be coupled with a pump supplying a fluid flow to the fluid inlet.

The cell support insert may be in the form of a disk. The cell support insert may be a carrier configured to support microcarrier beads or culture substrates. The bioreactor may include a plurality of cell support inserts removably disposed within the vessel.

According to an embodiment, the fluid inlet may be on the lid. Further, the lid may include the fluid distributor. The fluid distributor may form a seal against the interior surface of the vessel. The vessel may have a cylindrical shape. According to an embodiment, the fluid distributor has a top wall with an opening and an opposing bottom wall with a plurality of openings forming the plurality of fluid delivery ports. The opening in the top wall may define the fluid inlet. The plurality of fluid delivery ports may include 3 or more, 5 or more, 7 or more, 10 or more, or 12 or more ports. The plurality of fluid delivery ports may provide 1 port per every 10 cm² or less, preferably 1 port per every 6 cm² or less of the area of the cell growth insert perpendicular to a longitudinal axis of the vessel. The fluid distributor may be spaced apart from the cell support insert by a distance of 8 mm or greater, 10 mm or greater, or 12 mm or greater, and/or by a distance of 30 mm or less, 20 mm or less, or 15 mm or less. In some embodiments, the fluid distributor is spaced apart from the cell support insert by a distance of 8 mm to 30 mm or 10 mm to 20 mm.

According to an embodiment, a bioreactor system includes the bioreactor; a pump operatively coupled with the bioreactor fluid inlet; and a volume of cell culture media. The bioreactor system may be a closed system.

According to an embodiment, a method of culturing cells includes applying cell culture media and prepared cells to the vessel of the bioreactor of any one of the preceding claims; and circulating the cell culture media from the fluid outlet to the fluid inlet. The cell culture media may be circulated at a rate of 25 mL/min or greater, 50 mL/min or greater, 60 mL/min or greater, or 70 mL/min or greater, and 125 mL/min or less, 100 mL/min or less, or 80 mL/min or less, or about 75 mL/min. The circulating of the cell culture media applies a shear force of 0.15 Pa or less, 0.12 Pa or less, or 0.1 Pa or less, and 0.005 Pa or greater, 0.008 Pa or greater, or 0.1 Pa or greater, to the cells.

### BRIEF DESCRIPTION OF FIGURES

FIG. 1 is a schematic perspective view of a bioreactor in a bioreactor system according to an embodiment.

FIGS. 2A and 2B are schematic representations of flow patterns and shear stress patterns within the bioreactor of FIG. 1 according to an embodiment.

FIG. 2C is a segment of the shear stress pattern shown in FIG. 2B.

FIG. 3A is a schematic perspective view of the bioreactor of FIG. 1 according to an embodiment.

FIG. 3B is a schematic cross-sectional view of the bioreactor of FIG. 1 according to an embodiment.

FIG. 4 is a schematic cross-sectional view of a fluid distributor of the bioreactor of FIG. 1 according to an embodiment.

FIG. 5A is a schematic perspective view of a top portion of the fluid distributor of FIG. 4 according to an embodiment.

FIG. 5B is a schematic cross-sectional view of the top portion of FIG. 5A.

FIG. 5C is a plan view of the top portion of FIG. 5A.

FIG. 6A is a schematic perspective view of a bottom portion of the fluid distributor of FIG. 4 according to an embodiment.

FIG. 6B is a schematic cross-sectional view of the bottom portion of FIG. 6A.

FIG. 6C is a plan view of the bottom portion of FIG. 6A.

FIG. 7A is a schematic perspective view of a fluid distributor for the bioreactor of FIG. 1 according to an embodiment.

FIG. 7B is an exploded view of the fluid distributor of FIG. 7A.

FIG. 8A is a schematic top view of a cell support insert for the bioreactor of FIG. 1 according to an embodiment.

FIG. 8B is a schematic side view of the cell support insert of the FIG. 8A.

FIG. 8C is a schematic plan view of the cell support insert of the FIG. 8A inside the bioreactor of FIG. 1.

FIG. 9A is a schematic perspective view of another cell support insert for the bioreactor of FIG. 1 according to an embodiment.

FIG. 9B is a schematic cross-sectional side view of the cell support insert of the FIG. 9A.

FIG. 9C is a schematic cross-sectional side view of the cell support insert of the FIG. 9A inside a bioreactor according to an embodiment.

FIG. 10, views A-I are graphical representations of simulated shear stress patterns of fluid distributors according to Example 1.

FIGS. 11-15C are graphical representations of data from Example 2.

FIG. 16 is a microscopic image of Alizarin Red dyed cells cultured in Example 2.

FIGS. 17A-17C are graphical representations of data from Example 2.

### Definitions

All scientific and technical terms used herein have meanings commonly used in the art unless otherwise specified. The definitions provided herein are to facilitate understanding of certain terms used frequently herein and are not meant to limit the scope of the present disclosure.

The term “substantially” as used here has the same meaning as “significantly,” and can be understood to modify the term that follows by at least about 90%, at least about 95%, or at least about 98%.

The term “about” is used here in conjunction with numeric values to include normal variations in measurements as expected by persons skilled in the art, and is understood to have the same meaning as “approximately” and to cover a typical margin of error, such as +5% of the stated value.

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

Any direction referred to here, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

### DETAILED DESCRIPTION

The present disclosure relates to bioreactors and bioreactor systems for the expansion and cultivation of cells, such as stem cells. The present disclosure further relates to bioreactors that can support dynamic cell culture conditions for 2D and 3D cell cultures. The present disclosure also relates to bioreactors and bioreactor systems for the production of exosomes and exosome-rich cultures.

Conventionally, stem cells have been grown on two-dimensional surfaces, such as T-flasks or well-plates. As cell therapies continue to be developed, these systems have been scaled out into cell flask factories that include multiple T-flasks to accommodate the culturing of increased numbers of cells. This method of cell expansion has been notably associated with autologous cell therapy, in which patients are both the cell source and recipient. However, a more overarching form of cell therapy, allogenic treatment for which a patient receives cells from another patient donor, adopts a scaled-up production methodology that employs larger volume culturing systems (bioreactors) to support clinical needs.

Bioreactors are used for growing large numbers of cells or preparing cells in vitro for potential in vivo application. The application of dynamic forces on cells during culture-such as they would experience in vivo, is thought to lead to a plethora of enhanced properties, one of which is an increase in secreted products that have also shown promising applications and great therapeutic benefits. The present disclosure provides bioreactors and bioreactor systems designed to apply a dynamic force during cell culturing to stimulate the cells towards increasing production of cell-secreted products. The bioreactors and bioreactor systems of the present disclosure are suitable for the expansion and cultivation of many types of cells, including stem cells such as adult/somatic stem cells (ASCs), embryonic stem cells (ESCs) and induced pluripotent stem cells (iPSCs). The bioreactors and bioreactor systems of the present disclosure are suitable for the expansion and cultivation of human mesenchymal stromal/stem cells (MSCs), which are one type of ASC. The bioreactors and bioreactor systems of the present disclosure are also suitable for the expansion and cultivation of non-human cells, such as mouse cells, Chinese hamster ovary (CHO) cells, and the like.

Bioreactors are generally vessels or systems used to support biological and/or chemical processes or reactions. Currently, such systems are actively utilized in industries that involve fermentation, pharmaceuticals, food processing, and research, to name a few. There are various types of bioreactors currently in use within academia and industry. Typically, bioreactor designs are selected based on the specific application, cell type of choice, and preferred parameters, such as achieving desired working volume, product yield, cell yield, flow, and aggregation. The designs may offer combinations of various micro-environmental parameters to induce a specific combination of desired factors (increased proliferation, differentiation potential, etc.). However, there is still a need for improvement in the field, and a need for bioreactors that are capable of applying a specific or desired hydrodynamic flow, shear stress magnitude and duration, and the time points at which these parameters are applied to the cell culture. Further, there is a need for bioreactors that are capable of better mimicking the in vivo environment.

In the present disclosure, bioreactors and bioreactor systems are provided that are capable of a specific or desired hydrodynamic flow, shear stress magnitude and duration, and the time points at which these parameters are applied to the cell culture. Further, there bioreactors and bioreactor systems are provided that are capable of mimicking the in vivo environment.

The bioreactors and bioreactor systems of the present disclosure may be used to produce exosome-rich cultures, from which exosomes may be separated and harvested.

Exosomes are extracellular vesicles (EVs) that are secreted through the release of intracellular vesicles. They are secreted when multivesicular bodies fuse with the cell membrane. Exosomes are nanometer-sized EVs, around 40 to 150 nm, that primarily function in cell-to-cell communication, and are able to carry an abundance of factors such as cytokines, growth factors, signaling lipids, mRNAs and miRNAs. MSC-derived exosomes have been researched as a potential cell-free alternative to cell therapies. They have low immune-rejection, perfuse through the blood-brain barrier, have anti-inflammatory properties, and are non-tumorous. Exosomes have shown promise in regulating immune response in various diseases such as multiple sclerosis, fibrosis, stroke, cardiovascular disease, and other ischemic disease conditions. While the exosomes themselves have various properties, they can also be used as a drug delivery systems housing anti-cancer, anti-fungal, and analgesic components. Exosomes are typically characterized by size, shape, density, expressed proteins, lipids, and mi/mRNAs, and typically have a density of about 1.13 g/mL to 1.19 g/mL. Through various processes, exosomes can be isolated and extracted from cell media (conditioned media) or other fluids containing exosomes. Currently, ultracentrifugation and ultracentrifugation coupled with density gradient centrifugation are two techniques used to isolated exosomes from conditioned media.

In the present disclosure, bioreactors and bioreactor systems are described that are suitable for the expansion and cultivation of cells and for the production of exosomes and exosome-rich cultures.

According to an embodiment, the bioreactor includes a vessel with a wall, the wall having an interior surface defining a volume. The vessel includes a fluid inlet and a fluid outlet. A cell support insert is removably disposed within the vessel. The cell support insert is interchangeable and a user may select a suitable insert for the intended purpose. The bioreactor includes a lid constructed to close the vessel. According to an embodiment, the lid forms the top wall of the vessel. The vessel may have a bottom wall. The bottom wall may be integral or permanently attached to the side wall of the vessel. The bioreactor includes a fluid distributor in fluid communication with and arranged to receive a fluid flow from the fluid inlet. According to an embodiment, the fluid distributor has a plurality of fluid delivery ports constructed to deliver fluid onto the cell support insert (e.g., onto cells being cultured on the cell support insert). The fluid distributor may be configured to produce desired fluid flow dynamics and shear stress experienced by the cells being cultured on the cell support insert.

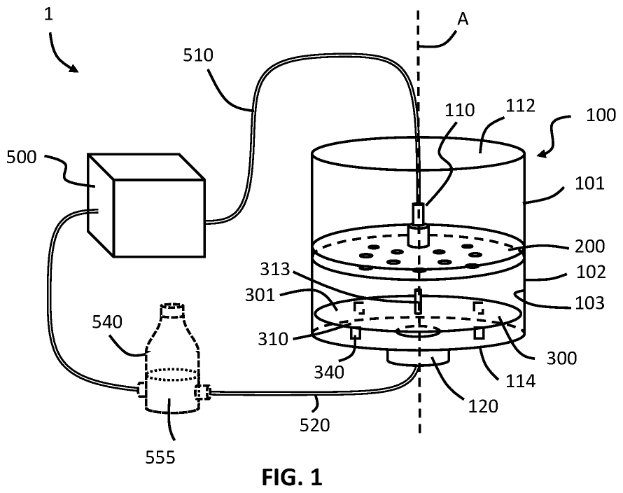

Referring now to FIG. 1, a bioreactor system 1 including a bioreactor 100 and a pump 500 is shown. According to an embodiment, the bioreactor system 1 may be a closed system, arranged to circulate cell culture media 555 to and from the bioreactor 100. The bioreactor system 1 may include a volume of cell culture media 555. The pump 500 may be operatively connected to the bioreactor 100 via an inlet line 510 and an outlet line 520. The pump 500 may be configured to provide a desired flow rate of cell culture media 555 to the bioreactor 100. For example, the pump 500 may be arranged to deliver fluid to the fluid distributor at flow rates between 25 mL/min and 125 mL/min. The bioreactor system 1 may optionally include a media reservoir 540 to house excess cell culture media 555. The media reservoir 540 may be used to facilitate long-term cultures, e.g., longer than 14 days.

According to an embodiment, the bioreactor 100 includes a vessel 101. The vessel 101 has a wall 102 defining an inside surface 103. The wall 102 may be a cylindrical wall, as shown. Other shapes are also possible, but for practical reasons, a cylindrical wall is preferred. According to an embodiment, the cylindrical wall 102 of the vessel 101 is an upright cylinder having a longitudinal axis A arranged vertically. The bioreactor includes a lid 200 constructed to close the vessel 101. According to an embodiment, the lid 200 forms the top wall of the vessel 101. The vessel 101 also has a bottom wall 114. The bottom wall 114 may be integral or permanently attached to the side wall 102 of the vessel 101.

The vessel 101 has in inlet 110. The inlet 110 may be on the top side (e.g., attached to the top wall 200) of the vessel 101, as shown. The vessel 101 also has an outlet 120. The outlet 120 may be at the bottom (e.g., part of the bottom wall 114) of the vessel 101, as shown. The inlet line 510 from the pump 500 is connected to (in fluid communication with) the inlet 110. The outlet 120 of the vessel 100 is connected to (in fluid communication with) the pump 500 via the outlet line 520. The inlet 110, outlet 120, or both the inlet 110 and the outlet 120 may be positioned coaxially with the cylindrical wall 102 of the vessel 101. Alternatively, the inlet 110, outlet 120, or both the inlet 110 and the outlet 120 may be positioned off center, away from the center axis A of the vessel 101.

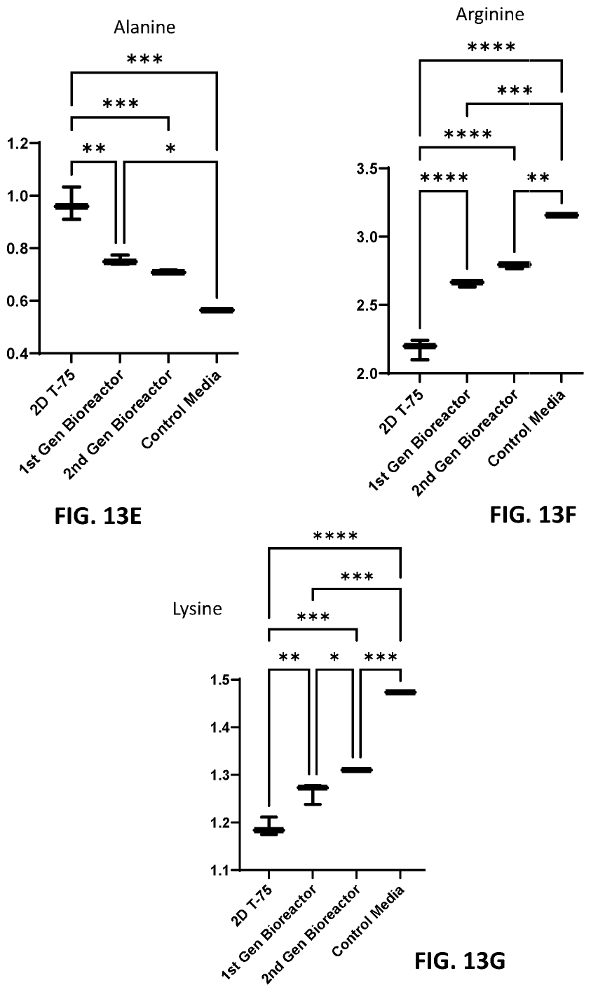

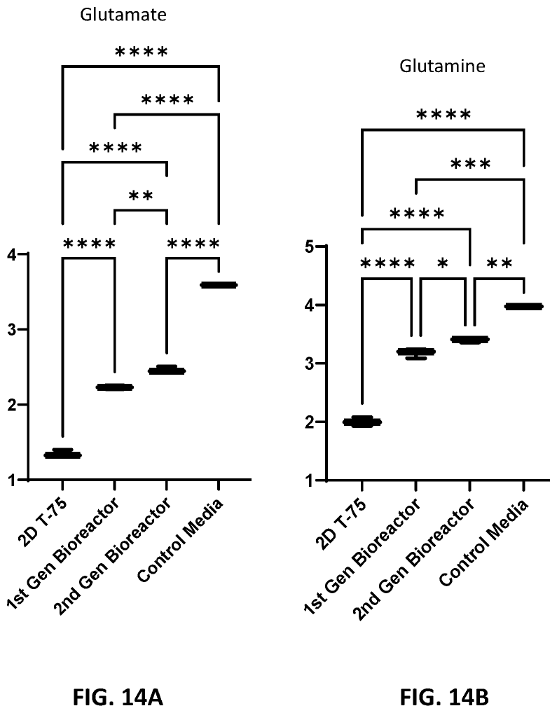

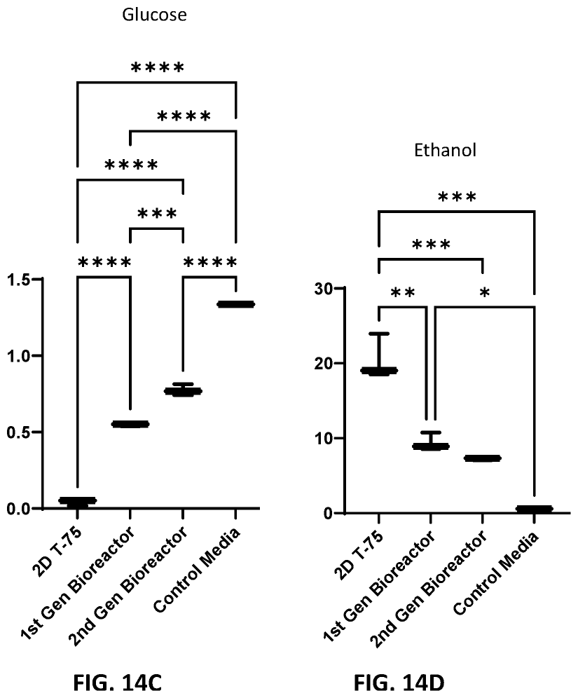

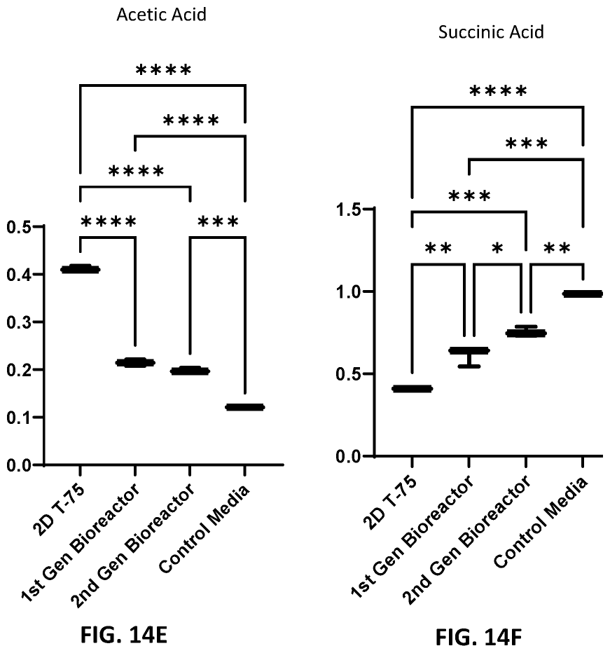

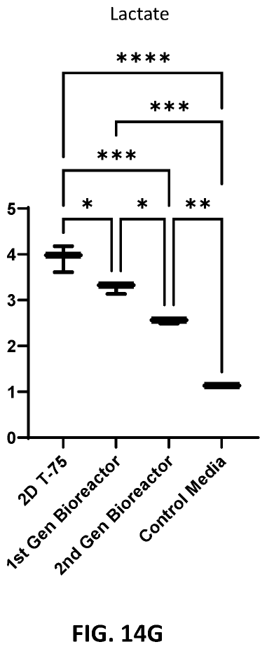

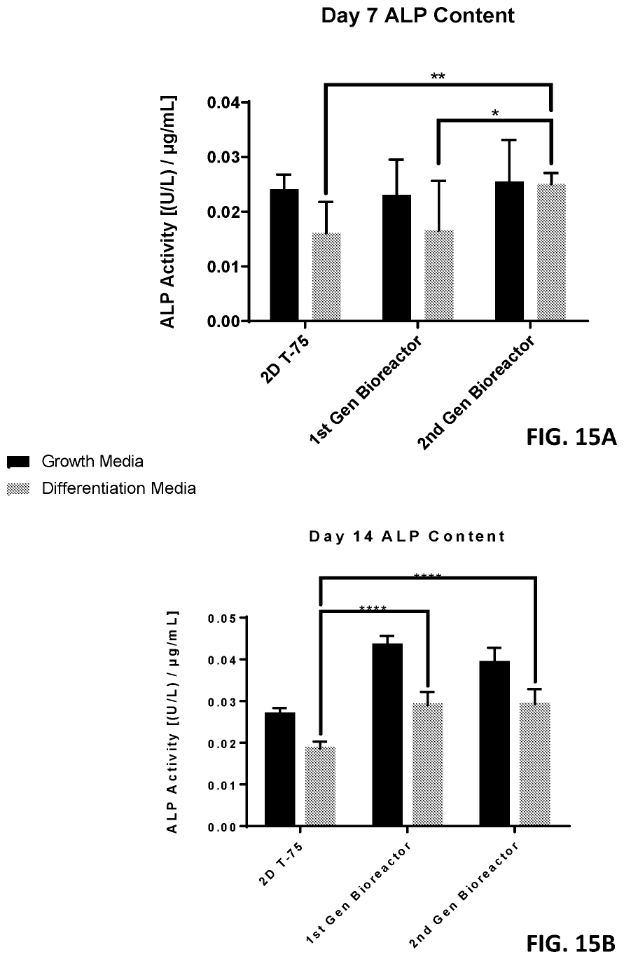

According to an embodiment, a cell support insert 300 is removably disposed within the vessel 101. The cell support insert 300 may be arranged adjacent the bottom 114 of the vessel 101. The cell support insert 300 may be spaced away from the bottom 114 by spacers 340. The spacers 340 may facilitate fluid flow below the cell support insert 300, from the interior of the vessel 101 into the outlet 120. The cell support insert 300 is configured for expansion and/or cultivation of cells, such as stem cells. The cell support insert 300 may be configured for expansion and/or cultivation of anchorage-dependent cells. Various shapes and sizes of cell support inserts 300 may be used in the bioreactor 100. In FIG. 1, the cell support insert 300, 310 is a simple flat disk with a handle 313. The cell support insert 300 may be sized so that fluid can flow between the edges of the cell support insert 300 and the wall 102 of the vessel 101. Alternatively or in addition, the cell support insert 300 may include one or more through openings to allow fluid to flow to the outlet 120. An exemplary cell support insert (carrier) 320 with openings 322 is shown in FIGS. 9A-9C. The cell support insert 300 is interchangeable and a user may select a suitable insert (e.g., insert 310 in FIGS. 8A-8C or insert 320 in FIGS. 9A-9C) for the intended purpose.

According to an embodiment, the bioreactor 100 includes a fluid distributor 210. The fluid distributor 210 is in fluid communication with and arranged to receive a fluid flow from the fluid inlet 110 and to distribute the fluid flow onto the cells being cultivated on the cell support insert 300. According to an embodiment, the fluid distributor 210 has a plurality of fluid delivery ports 202 constructed to deliver fluid onto the cells on the cell support insert 300. The fluid delivery ports 202 of the fluid distributor 210 are arranged to achieve a desired hydrodynamic flow on the cells. The fluid delivery ports 202 of the fluid distributor 210 are arranged to achieve a desired level of shear stress on the cells. The hydrodynamic flow and shear stress may be selected based on the desired growing conditions and outcome of the expansion and/or cultivation. According to an embodiment, the bioreactor 100 does not include any other means of agitation. For example, the bioreactor 100 may be free of any impellers or mixers. The vessel 101 of the bioreactor 100 may be configured to be static, e.g., the vessel 101 is not rotated, shaken, or vibrated during cultivation.

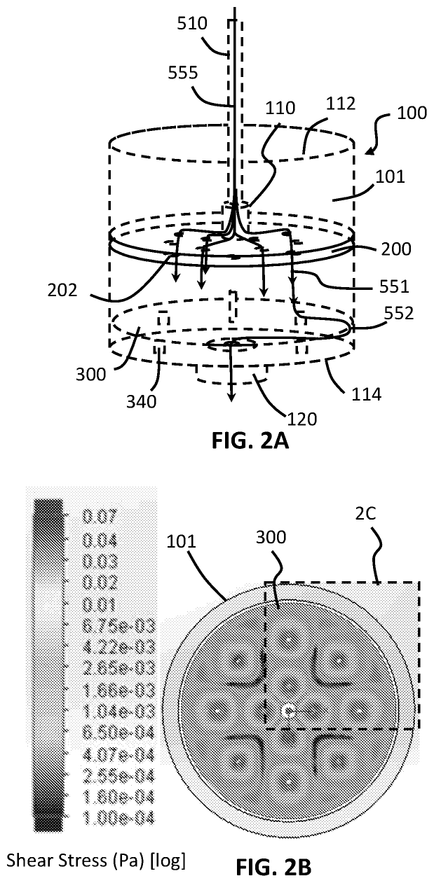

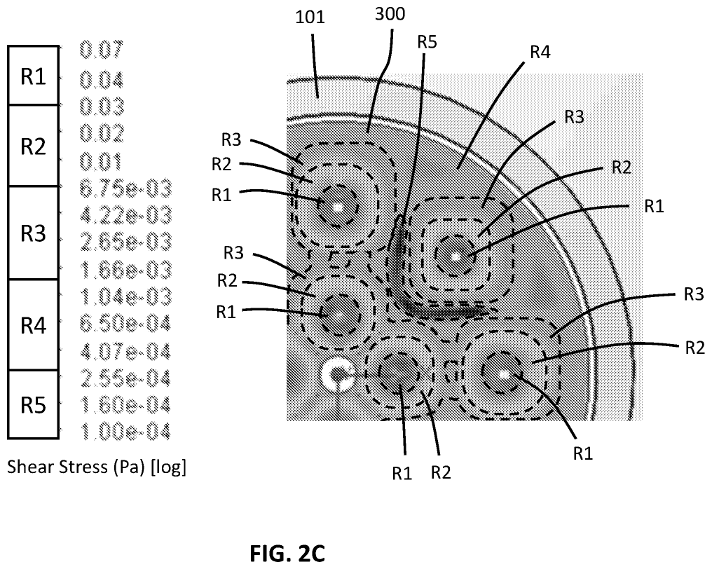

An exemplary flow pattern and resulting shear stress pattern are schematically illustrated in FIGS. 2A, 2B, and 2C. The cell culture media 555 is flown into the bioreactor 100 through the inlet 110 and is distributed through the plurality of fluid ports 202 in a fluid flow pattern using the fluid distributor 210. According to an embodiment, the fluid flow is directed down (e.g., straight down) from the fluid ports 202 of the fluid distributor 210, as shown by arrows 551. The fluid flow may be applied using gravity flow and pressure from the pump 500, as desired. Arrow 552 shows the fluid flow around the sides of the cell support insert 300 to the outlet 120.

The fluid ports 202 may be disposed along the fluid distributor 210 in a pattern that results in the desired shear stress pattern. The fluid ports 202 may be evenly dispersed along the fluid distributor 210. The fluid ports 202 may be placed so that the distance between adjacent fluid ports 202 is greater than a minimum distance and smaller than a maximum distance. For example, the adjacent fluid ports 202 may be placed at a distance of 0.5 cm or greater, 0.8 cm or greater, 1 cm or greater, or 1.5 cm or greater from each other. Adjacent fluid ports 202 may be placed at a distance of 5 cm or less, 4 cm or less, or 3 cm or less from each other. The fluid ports 202 may be thought to cover a certain amount of surface area of the fluid distributor 210 and cell support insert 300. The number of fluid ports 202 may be selected based on the desired coverage. For example, a fluid distributor 210 having an area of about 50-60 cm² may have 3 or more, 5 or more, 7 or more, 10 or more, or 12 or more fluid ports 202. The fluid distributor 210 having an area of about 50-60 cm² may have 50 or fewer, 40 or fewer, 30 or fewer, or 20 or fewer fluid ports 202. In some embodiments, the plurality of fluid delivery ports 202 may include 1 port per every 10 cm² or less of the area of the fluid distributor 210, or 1 port per every 8 cm² or less of the area, preferably 1 port per every 6 cm² or less of the area. In the specific embodiment shown in FIG. 2B, the fluid distributor 210 includes 1 port per approximately every 4.5 cm².

FIG. 2C is a segment of the heat map shown in FIG. 2B and shows the intensity of the shear stress illustrated by color in the original heat map of FIG. 2B, as regions R1-R5 in FIG. 2C.

The diameter (hole size) of the fluid ports 202 may also be selected to adjust the flow rate and shear stress as desired. It has been found that as the diameter decreases, the maximum shear stress experienced by the cells increases and the distribution of shear stress becomes more localized. The diameter of the fluid ports 202 may also be selected in conjunction with the number of fluid ports 202 to accommodate the desired flow rate. According to an embodiment, the fluid ports 202 may have a diameter of 1 mm or greater, 1.25 mm or greater, 1.5 mm or greater, 1.75 mm or greater, or 2 mm or greater. The fluid ports 202 may have a diameter of 5 mm or less, 4 mm or less, 3 mm or less, 2.5 mm or less, or 2.25 mm or less. In some embodiments, the fluid ports 202 have a diameter between 1.5 mm and 2.5 mm.

According to an embodiment, the fluid distributor 210 is arranged to apply a certain shear force to the cells when cell culture media is circulated in the system. The shear force applied by the fluid distributor 210 may be adjusted by adjusting the number, size, and distribution of fluid ports 202, and the flow rate of cell culture media. For example, the fluid distributor 210 may be arranged to apply a shear force in the range 0.005 Pa to 0.15 Pa to the cells.

In some embodiments, the number, size, and distribution of fluid ports 202 are selected such that at flow rates between 25 mL/min to 125 mL/min, the cell culture media applies a shear force of 0.005 Pa to 0.15 Pa to the cells. The fluid distributor 210 and flow rate may be configured such that the shear force is between 0.008 Pa and 0.12 Pa or between 0.01 Pa and 0.1 Pa. FIG. 2B demonstrates a configuration where at a flow rate of 75 mL/min, the shear force ranges from 0.0001 Pa to 0.07 Pa across the surface of the cell support insert 300. Most of the area of the cell support insert 300 experiences a shear stress between 0.005 Pa and 0.07 Pa.

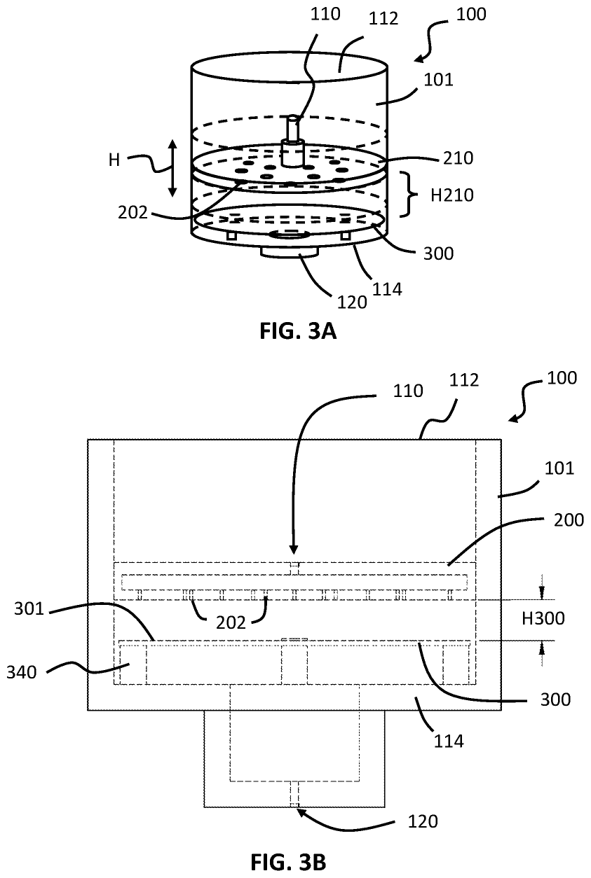

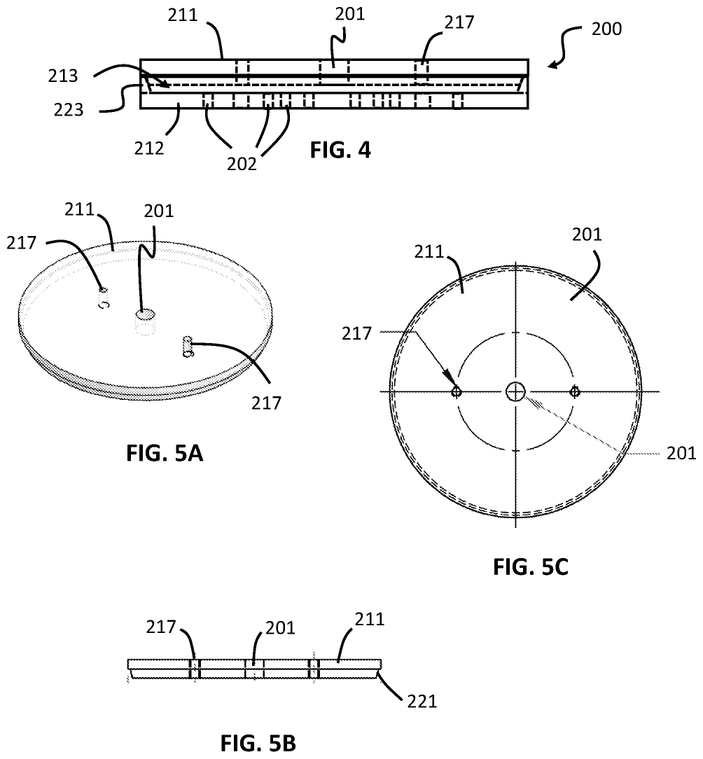

According to an embodiment, the fluid distributor 210 has an adjustable height, as indicated by the arrow H in FIG. 3A. The fluid distributor 210 has a height H210 from the cell support insert 300. Adjusting the height H210 allows a user to adjust the shear stress experienced by cells on the cell support insert 300. It has been found that as the distance is increased, the maximum shear stress decreases, and the overall distribution of shear stress is more uniform. It may be desirable to place the fluid distributor 210 at a height H210 of 8 mm or greater, 10 mm or greater, or 12 mm or greater from the cell support insert 300 (e.g., from the top surface of the cell support insert 300). The fluid distributor 210 may be placed at any height above the cell support insert 300, within the vessel 101. The fluid distributor 210 is spaced apart from the cell support insert 300 by a distance (height H210) of 100 mm or less, 30 mm or less, 20 mm or less, or 15 mm or less. In some embodiments, the fluid distributor 210 is spaced apart from the cells (e.g., from the cell support, the microcarrier beads, or other substrate or scaffold) by a distance (height H210) of 4 mm to 100 mm, 8 mm to 30 mm, or 10 mm to 20 mm.

In some embodiments, the fluid distributor 210 forms the lid 200 of the bioreactor 100. The lid 200 may further include the fluid inlet 110. In some embodiments, the fluid distributor 210 is separate from the lid 200. In other words, the bioreactor 100 may include a separate lid 200 and fluid distributor 210. In some embodiments, the lid 200 may be attached to the vessel 101 by a friction fit. In some embodiments, the lid 200 may be attached to the vessel 101 by threading.

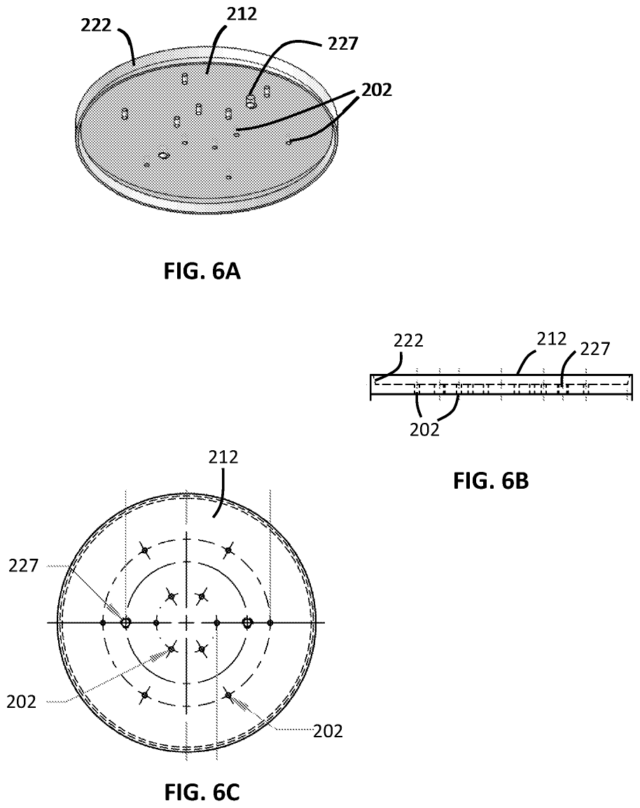

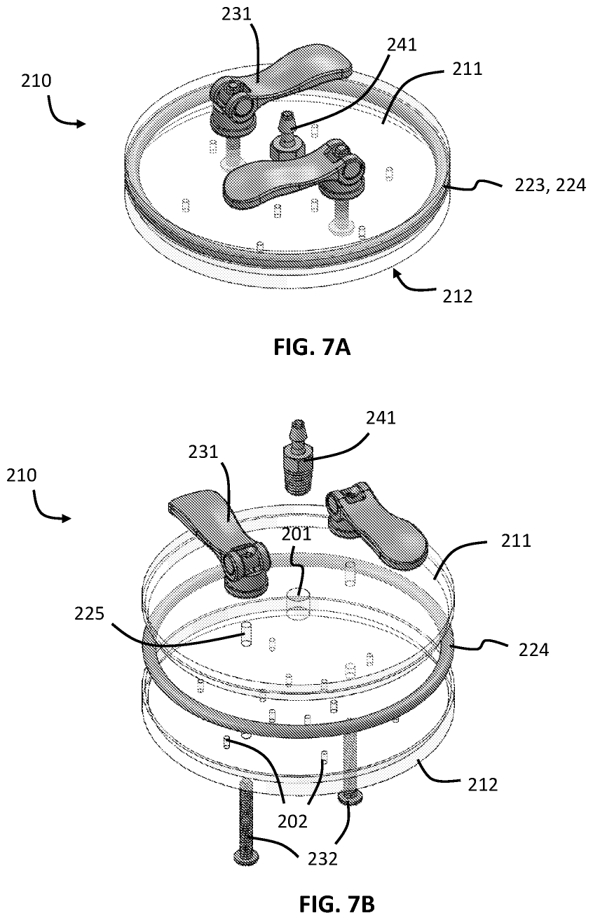

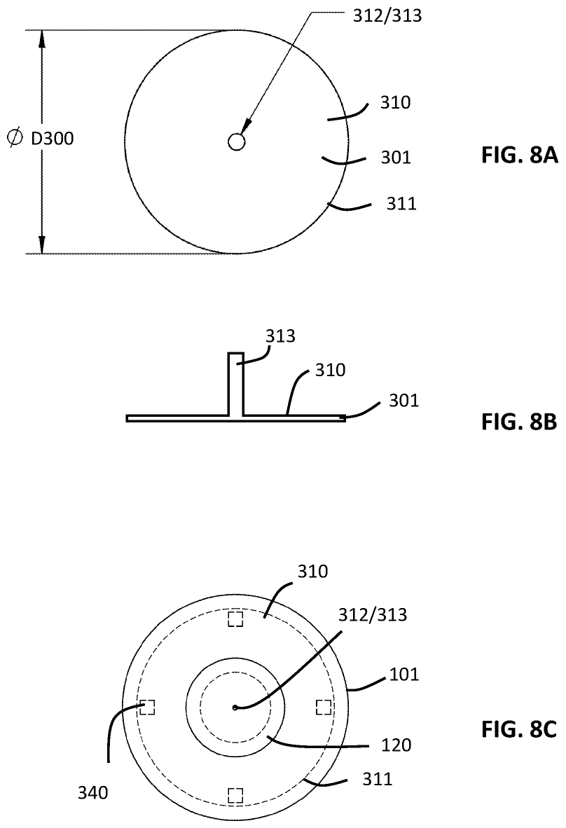

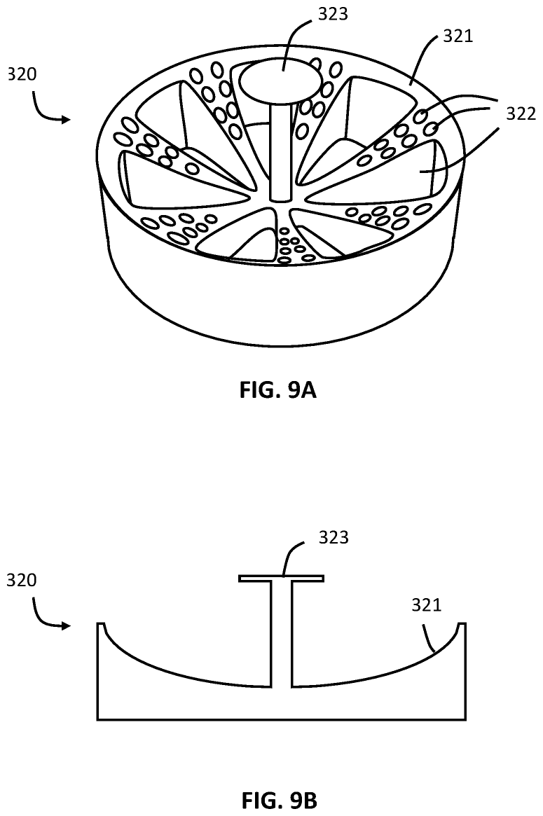

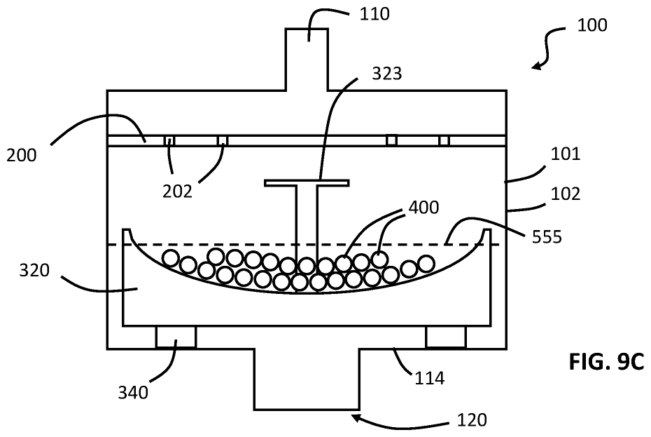

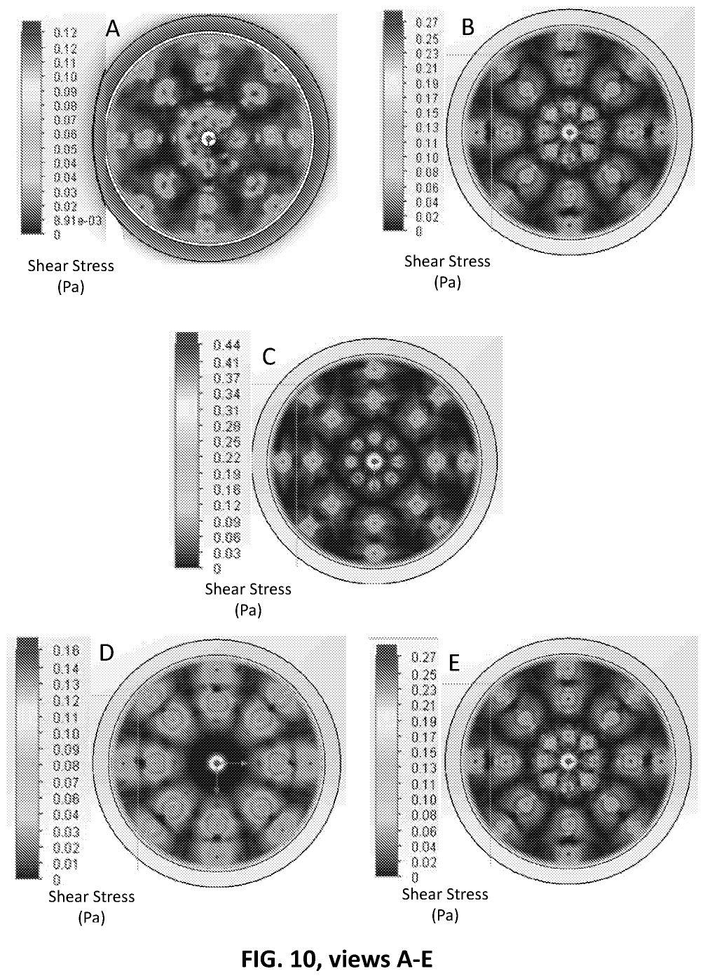

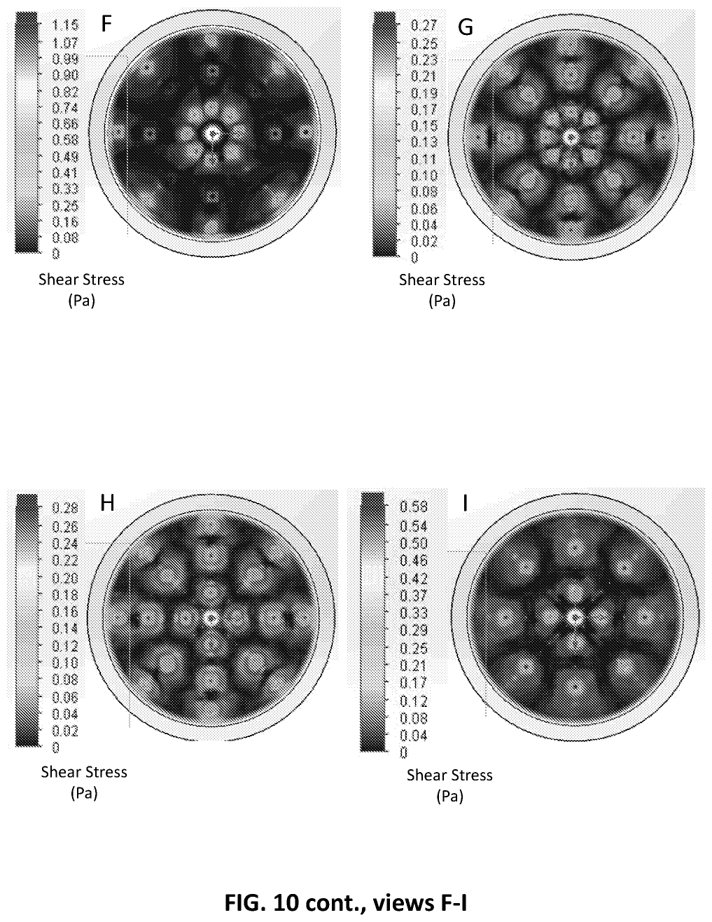

According to an embodiment, the fluid distributor 210 is formed as a hollow disk, shown in detail in FIGS. 4-7B. The fluid distributor 210 may include a top wall 211 and an opposing bottom wall 212. One or both of the top and bottom walls 211, 212 may include a lip or flange that couple together, forming a side wall 223 of the hollow disk and defining an interior space 213. In the embodiment shown, the bottom wall 212 includes a lip 222 that couples with a groove 221 on the top wall 211 to form the side wall 223. The side wall 223 may further be fitted with a seal 224 along its perimeter that seals the fluid distributor 210 against the inside surface 103 of the vessel 101.

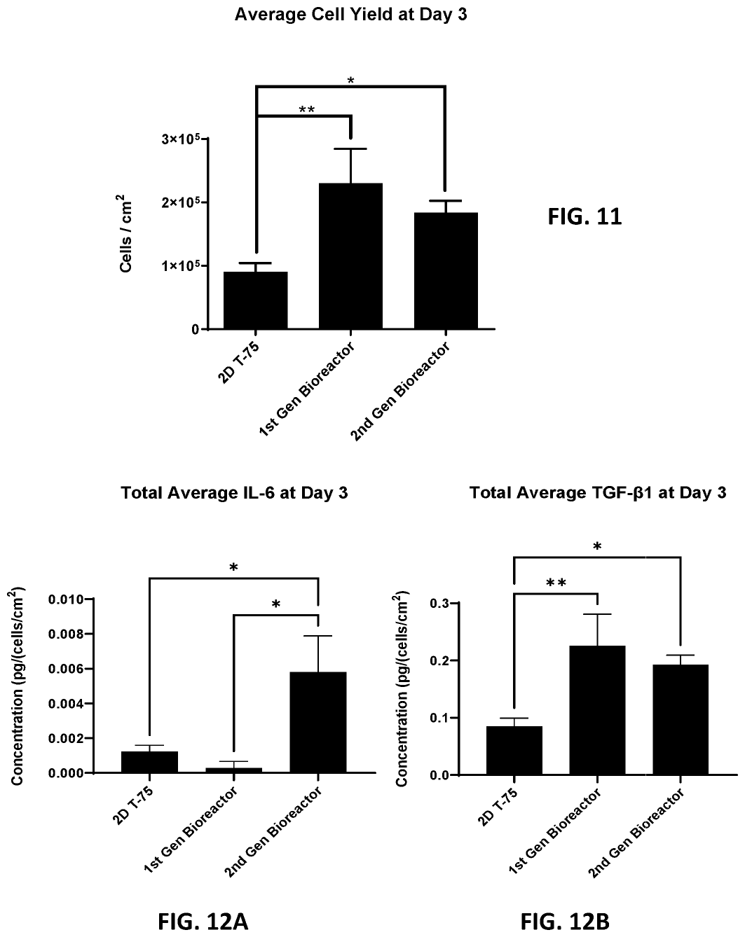

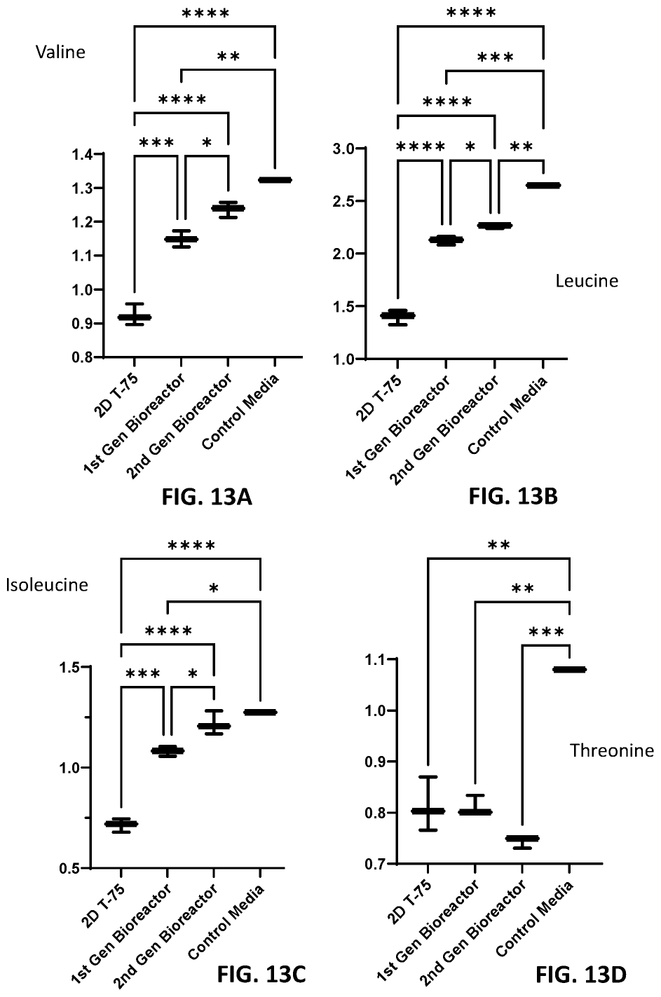

The top wall 211 includes a fluid inlet 201. The fluid inlet 201 may be fitted with a port 241. The top wall 211 may further include one or more openings 217 to facilitate attachment of one or more handles 231, such as the clamping handles shown in FIGS. 7A and 7B. The bottom wall 212 includes a plurality of fluid ports 202. The bottom wall 212 may further include one or more openings 227 to facilitate the attachment of the one or more handles 231.

The fluid distributor 210 may be formed using any suitable construction. In the exemplary embodiment shown in FIGS. 1-7B, the fluid distributor 210 is formed as a hollow disk with a plurality of openings on its underside to provide the plurality of fluid ports 202. However, alternative configurations can be envisioned that allow fluid to be delivered through multiple ports spaced at desired intervals. For example, the fluid distributor 210 may include a branched fluid conduit beginning at an inlet and terminating at a plurality of outlets.

Exemplary cell support inserts 300 are shown in FIGS. 8A-8C and 9A-9C. In FIGS. 8A-8C, the cell support insert 300 is a planar or substantially planar disk 301. The disk 301 has a top surface 310. The top surface 310 facilitates attachment and growth of cells. The cell support insert 300 may include a central opening 312 or may include a handle 313 extending from the disk 301. The cell support insert 300 has a diameter D300. The diameter D300 may be slightly smaller than the inside diameter of the vessel 101. In some embodiments, the diameter D300 is smaller than the inside diameter of the vessel 101 to facilitate fluid flow between the outer edge 311 of the disk 301 and the wall 102 of the vessel 101. In some embodiments, the cell support insert 300 includes one or more openings through the insert to facilitate fluid flow to below the cell support insert 300. In such embodiments, the diameter D300 of the cell support insert 300 may be substantially the same as the inside diameter of the vessel 101, or may be smaller. The cell support insert 300 may be placed on top of supports or spacers 340 to allow for fluid flow between the cell support insert 300 and the bottom wall 114 of the vessel 101.

FIGS. 9A-9C shown another embodiment of the cell support insert 300 that is configured as a carrier 320 for use with microcarrier beads 400 or other culture substrates or scaffolds. The carrier 320 may have a cup-shaped interior surface 321 and a plurality of openings 322 extending therethrough. The carrier 320 may further include a handle 323 extending from the interior surface 321. The handle 323 may be removable. FIG. 9C shows a schematic cross-sectional view of the carrier 320 in use in a bioreactor 100. The carrier 320 is disposed below the fluid distributor 210 and is loaded with microcarrier beads 400. The openings 322 in the carrier 320 may be sized so that the microcarrier beads 400 do not fall through, or an additional layer (e.g., a mesh or scaffold, e.g., a nylon mesh) may be placed on the carrier 320 to allow the microcarrier beads 400 to remain on top of the carrier 320. The microcarrier beads 400 may form a loose packed bed on the carrier 320. The carrier 320 may be placed on top of supports or spacers 340 to allow for fluid flow between the carrier 320 and the bottom wall 114 of the vessel 101.

In some embodiments, the bioreactor 100 may include a plurality of removable cell support inserts 300. The plurality of cell support inserts 300 may be stacked within the vessel.

The parts of the bioreactor 100 may be made from any suitable materials. In some embodiments, the parts of the bioreactor 100 are constructed from plastic, glass, ceramic, metal, or a combination thereof. Additional materials may also be possible. It may be desirable to construct at least the vessel from a transparent material, such as a transparent plastic (e.g., polycarbonate) or glass.

According to an embodiment, the cell support insert 300 may be treated or coated with another material. For example, the cell support insert 300 may be coated with a hydrogel. The hydrogel may include one or more compounds that may be released during use. Such hydrogel coatings may be used to culture cells in serum-free conditions.

A method of culturing cells in the bioreactor 1 may include applying cell culture media and prepared cells to the vessel 101 of the bioreactor 100. The cells may be applied directly on top of the cell support insert 300 or onto microcarrier beads 400 or another cell culture substrate or scaffold carried by a carrier 320. The method further includes circulating the cell culture media from the fluid outlet 120 to the fluid inlet 110, and distributing the cell culture media onto the cells through the fluid distributor 210. The cell culture media may be circulated at a rate of 25 mL/min or greater, 50 mL/min or greater, 60 mL/min or greater, or 70 mL/min or greater, and 125 mL/min or less, 100 mL/min or less, or 80 mL/min or less, or about 75 mL/min. Circulating of the cell culture media may apply a shear force of 0.15 Pa or less, 0.12 Pa or less, or 0.1 Pa or less to the cells. Circulating of the cell culture media may apply a shear force of 0.005 Pa or greater, 0.008 Pa or greater, or 0.1 Pa or greater to the cells. After a period of culturing and/or expansion, the cells, the conditioned media (including any exosomes), or both the cells and the conditioned media, may be harvested. Exosomes may be further separated from the harvested media.

### EXEMPLARY EMBODIMENTS

The following is a list of exemplary embodiments according to the present disclosure.

Embodiment 1 is a bioreactor comprising: a vessel comprising a wall with an interior surface defining a volume, and a fluid outlet; a cell support insert removably disposed within the vessel; a lid constructed to close the vessel; a fluid inlet; and a fluid distributor in fluid communication with and arranged to receive a fluid flow from the fluid inlet, the fluid distributor comprising a plurality of fluid delivery ports constructed to deliver fluid onto the cell support insert.

Embodiment 2 is the bioreactor of embodiment 1, wherein the fluid inlet is on the lid.

Embodiment 3 is the bioreactor of embodiment 1 or 2, wherein the fluid inlet is coupled with a pump supplying a fluid flow to the fluid inlet. The fluid inlet may be coupled with the pump via an inlet line (e.g., tubing).

Embodiment 4 is the bioreactor of any one of the preceding embodiments, wherein the cell support insert comprises a disk. The cell support insert may be a planar or substantially planar disk. The cell support insert may be a polycarbonate disk.

Embodiment 5 is the bioreactor of any one of the preceding embodiments, wherein the cell support insert comprises a carrier configured to support microcarrier beads or culture substrates. The carrier may comprise a cup-shaped interior surface. The carrier may comprise a plurality of openings extending through the carrier. The carrier may include a handle extending from the interior surface. The handle may be removable.

Embodiment 6 is the bioreactor of any one of the preceding embodiments, wherein the lid is coupled with the vessel via screw threading.

Embodiment 7 is the bioreactor of any one of embodiments 1 to 5, wherein the lid is coupled with the vessel via friction fit. The lid may include a seal along its perimeter.

Embodiment 8 is the bioreactor of any one of the preceding embodiments, wherein the vessel has a cylindrical shape.

Embodiment 9 is the bioreactor of any one of the preceding embodiments comprising a plurality of cell support inserts removably disposed within the vessel.

Embodiment 10 is the bioreactor of embodiment 8, wherein the plurality of cell support inserts are stacked within the vessel.

Embodiment 11 is the bioreactor of any one of the preceding embodiments, wherein the lid comprises the fluid distributor.

Embodiment 12 is the bioreactor of any one of the preceding embodiments, wherein the fluid distributor forms a seal against the interior surface of the vessel.

Embodiment 13 is the bioreactor of any one of the preceding embodiments, wherein the fluid distributor comprises a branched fluid flow channel comprising an inlet end and terminating at the plurality of fluid delivery ports.

Embodiment 14 is the bioreactor of any one of the preceding embodiments, wherein the fluid distributor comprises a top wall comprising an opening and an opposing bottom wall comprising a plurality of openings comprising the plurality of fluid delivery ports.

Embodiment 15 is the bioreactor of any one of the preceding embodiments, wherein the opening in the top wall defines the fluid inlet.

Embodiment 16 is the bioreactor of any one of the preceding embodiments, wherein the plurality of fluid delivery ports comprises 3 or more, 5 or more, 7 or more, 10 or more, or 12 or more ports.

Embodiment 17 is the bioreactor of any one of the preceding embodiments, wherein the cell growth insert defines an area perpendicular to a longitudinal axis of the vessel, and wherein the plurality of fluid delivery ports comprises 1 port per every 10 cm² or less of the area, preferably 1 port per every 6 cm² or less of the area. Adjacent fluid ports may be placed at a distance of 0.5 cm or greater, 0.8 cm or greater, 1 cm or greater, or 1.5 cm or greater from each other. Adjacent fluid ports may be placed at a distance of 5 cm or less, 4 cm or less, or 3 cm or less from each other.

Embodiment 18 is the bioreactor of any one of the preceding embodiments, wherein the fluid distributor is spaced apart from the cell support insert by a distance of 8 mm or greater, 10 mm or greater, or 12 mm or greater.

Embodiment 19 is the bioreactor of any one of the preceding embodiments, wherein the fluid distributor is spaced apart from the cell support insert by a distance of 30 mm or less, 20 mm or less, or 15 mm or less.

Embodiment 20 is the bioreactor of any one of the preceding embodiments, wherein the fluid distributor is arranged to apply a shear force of 0.15 Pa or less, 0.12 Pa or less, or 0.1 Pa or less, and 0.005 Pa or greater, 0.008 Pa or greater, or 0.1 Pa or greater, to the cells when fluid is circulated at a rate of 25 mL/min to 125 mL/min.

Embodiment 21 is a bioreactor system comprising: the bioreactor of any one of the preceding embodiments; a pump operatively coupled with the bioreactor fluid inlet; and a volume of cell culture media, wherein the bioreactor system is a closed system. The pump may be coupled with the fluid inlet via an inlet line. The bioreactor may include an outlet coupled with the pump via an outlet line. The inlet line, outlet line, or both the inlet line and outlet line may further comprise a fluid reservoir.

Embodiment 22 is a method of culturing cells, the method comprising: applying cell culture media and prepared cells to the vessel of the bioreactor of any one of the preceding embodiments; and circulating the cell culture media from the fluid outlet to the fluid inlet.

Embodiment 23 is the method of embodiment 22, wherein the cell culture media is circulated at a rate of 25 mL/min or greater, 50 mL/min or greater, 60 mL/min or greater, or 70 mL/min or greater, and 125 mL/min or less, 100 mL/min or less, or 80 mL/min or less, or about 75 mL/min.

Embodiment 24 is the method of embodiment 22 or 23, wherein the circulating of the cell culture media applies a shear force of 0.15 Pa or less, 0.12 Pa or less, or 0.1 Pa or less, and 0.005 Pa or greater, 0.008 Pa or greater, or 0.1 Pa or greater, to the cells.

### EXAMPLES

### Example 1

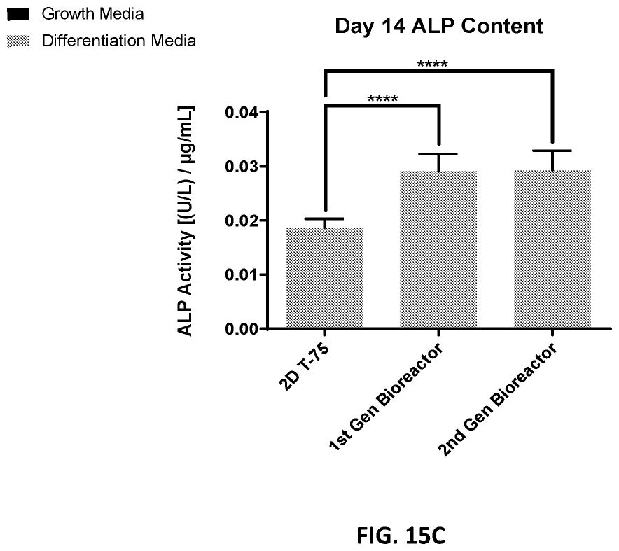

Various configurations of fluid ports and their impact on shear stress were simulated using SOLIDWORKSR (Dassault Systems, Velizy-Villacoublay, France). The overall diameter of the fluid distributor was 86.36 mm. The cell support insert was a flat disk. The flow rate was 300 mL/min and the temperature was 310 K. The parameters that were varied included the height of the fluid ports from the cell support (FIG. 10, view A at 16 mm, view B at 10 mm, and view C at 6 mm); fluid port hole diameter (FIG. 10, view D 3.18 mm, view E 1.58 mm, and view F 0.79 mm; maintaining the height at 10 mm for each); and number of fluid ports (FIG. 10, view G with 24 fluid ports, view H with 20 fluid ports, and view I with 12 fluid ports; maintaining the height at 10 mm and fluid port diameter at 1.58 mm for each). The shear stress at the cell support insert is shown in the heat maps of FIG. 10, views A-I. Generally, the area under each fluid port is seen in a lighter gray, while the areas between fluid ports are seen in a darker gray. The light gray and dark gray areas range in value as shown in TABLE A below.

TABLE A

| | Shear stress in light-colored | Shear stress in dark-colored |
|---|---|---|
| View | areas (Pa) | areas (Pa) |
| A | 0.04-0.09 | 0-0.03 |
| B | 0.07-0.2 | 0-0.06 |
| C | 0.1-0.34 | 0-0.9 |
| D | 0.03-0.11 | 0-0.02 |
| E | 0.06-0.2 | 0-0.05 |
| F | 0.25-1.0 | 0-0.2 |
| G | 0.06-0.2 | 0-0.05 |
| H | 0.06-0.25 | 0-0.05 |
| I | 0.08-0.5 | 0-0.07 |

### Example 2

Murine bone marrow-derived MSCs (DI CRL-124TM, ATCC® Manassas, VA) were cultured as a monolayer culture using T-flasks that are static (i.e., no dynamic force applied to cells) and a bioreactor configured with a single planar cell support insert and a single central flow inlet (“1^{st} generation”) and a multiple-inlet-port fluid distributor (“2^{nd} generation”).

The flasks, bioreactors, and other equipment were sterilized with 70% ethanol. The cell support inserts were sterilized with ethanol and plasma treated to remove organic particulates, increase wettability, and promote cell adhesion. Culture media was added to the vessels and the cell support inserts were placed in the bioreactors. The culture media was DMEM-Complete which consisted of low-glucose Dulbecco's Modified Eagle Medium (DMEM, Gibco, Waltham, MA), 10% Fetal Bovine Serum (FBS, Hyclone Laboratories Inc., Logan, UT), and 1% 10,000 U/mL Penicillin/Streptomycin (Gibco). Cells were then seeded onto the cell support inserts and the tops of the vessels were attached. The cells were seeded at 4,000,000 cells at passage numbers 12-15, within the T-75 flask, 1^{st} generation system, and 2^{nd} generation system.

The assembled 1^{st} generation bioreactor, 2^{nd} generation bioreactor, and T-75 flask were placed into an incubator at standard cell culture conditions of 37° C. and 5% CO₂. The two bioreactor systems were attached to a peristaltic pump (Single-channel MasterFlex L/S Digital Miniflex peristaltic pump, Cole-Parmer) and the tubing was primed with culture media. After approximately 18 hours of static incubation to allow the DI cells time to adhere to the system's monolayer insert, the peristaltic pump was turned on to initiate flow. Once flow was initiated, the flow rate was set to 1 mL/min for the 1^{st} generation system and to 75 mL/min for the 2^{nd} generation system. The conditions used for each system are shown in TABLE 1. Each of the cultures was run in triplicate.

TABLE 1
Culture Conditions.

| | | Cell | | | | Media | |
|---|---|---|---|---|---|---|---|
| | | Seeding | Pas- | Tem- | | Flow | |
| | Trial | Density | sage | per- | | Rate | Media |
| Culture | Num- | (Total | Num- | ature | % | (mL/ | Volume |
| Method | ber | Cells) | ber | (° C.) | CO₂ | min) | (mL) |
| T-75 | 1 | 4,000,000 | 12 | 37 | 5 | 0 | 10 |
| | 2 | | | | | | |
| | 3 | | | | | | |
| 1^{st} | 1 | 4,000,000 | 12 | 37 | 5 | 1 | 110 |
| Generation | 2 | | | | | | |
| Bioreactor | 3 | | | | | | |
| 2^{nd} | 1 | 4,000,000 | 15 | 37 | 5 | 75 | 120 |
| Generation | 2 | | 13 | | | | |
| Bioreactor | 3 | | 14 | | | | |

When harvesting cells at day 3, media samples of 10-15 mL were collected. After media collection, the cells were cleaved using trypsinization, centrifuged, and resuspended in DMEM-Complete. The cells were counted via hemocytometer. Average cell yield and standard deviation were calculated for each of the three trials and tabulated in TABLE 2.

The cell culture media samples were analyzed for metabolites and cytokines secreted into each system. The metabolites were analyzed by nuclear magnetic resonance (NMR) using Bruker NEO 800 MHZ NMR spectrometer (Bruker Corp. in Billerica, MA). A control sample was used that included media not in contact with any cells. Transforming Growth Factor-Beta 1 (TGB-β1) and Interleukin-6 (IL-6) were analyzed using a Mouse TGB-β1 ELISA Kit and Mouse IL-6 ELISA Kit, respectively, (both from ThermoFisher Scientific) and a BioTek 800TS microplate reader (BioTek Instruments, Winooski, VT).

The media was also analyzed to assess extracellular protein content after three days of MSC culture using a Pierce BCA Protein Assay Kit (Thermo Fisher Scientific) and the BioTek 800TS microplate reader. Total extracellular protein content was normalized using cell count data obtained from a hemocytometer.

Conditioned cells expanded in the 1^{st} generation system, 2^{nd} generation system, and T-75 flasks during the short-term study were subsequently evaluated using several assays to assess their ability to undergo osteogenic differentiation. Cells were cultured in DMEM-Complete growth media or Osteogenic Differentiation media consisting of High Glucose-Dulbecco's Modified Eagle Medium (Gibco), 10% Fetal Bovine Serum (Hyclone Laboratories Inc.), 1% 10,000 U/mL Penicillin/Streptomycin (Gibco), 50 μg/mL Ascorbic Acid (#A-8960, Sigma-Aldrich, St. Louis, MO), and 4 mM Beta-glycerophosphate (#G-5422, Sigma-Aldrich). The cells were assayed at days 7 and 14 post-osteogenic differentiation induction.

Total intracellular protein content for cells seeded for osteogenic induction was assessed at days 7 and 14 also using the Pierce BCA Protein Assay Kit.

Production of calcium-based mineral formation was evaluated at day 7 and day 14 following the introduction of osteogenic differentiation media at day 0. The cells were dyed with Alizarin Red for semi-quantitative analysis using the BioTek 800TS microplate reader. The absorbance data was then normalized using total intracellular protein concentration previously obtained with the Pierce BCA Protein Assay Kit.

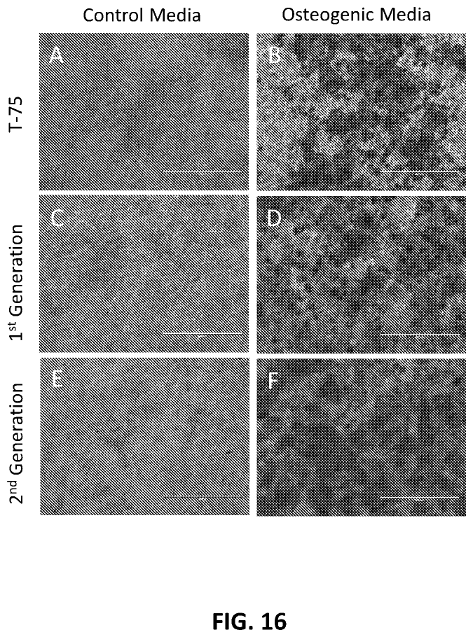

The results are shown in TABLE 2 and FIGS. 11-18C.

TABLE 2
Cell Yield per cm².

| | 2D T-75 Flask | 1st Gen. Bioreactor | 2nd Gen. Bioreactor |
|---|---|---|---|
| Trial 1 | 103,000 | 231,000 | 192,000 |
| Trial 2 | 92,800 | 284,000 | 197,000 |
| Trial 3 | 75,700 | 175,000 | 162,000 |
| Average | 90,500 | 230,000 | 184,000 |
| Standard | 13,900 | 54,800 | 18,900 |
| Deviation | | | |

FIG. 11 shows resulting cell yields post-expansion. Both bioreactor systems were found to support higher proliferation than the static culture in the 2D T-75 flask. Statistical difference is indicated in the chart with an asterisk (* for p≤0.05 and ** for p≤0.01).

FIGS. 12A and 12B show the total average IL-6 and TGF-β1, respectively.

Bioreactor cultures were found to result in significantly higher levels of each cytokine in comparison to the static flask cultures.

FIGS. 13A-13G show the valine, leucine, isoleucine, threonine, alanine, arginine, and lysine content, respectively, in conditioned media samples. Statistical difference is indicated in the charts with an asterisk (* for p≤0.05, ** for p≤0.01, *** for p≤0.001, and **** for p≤0.0001).

FIGS. 14A-14G show the metabolites glutamate, glutamine, glucose, ethanol, acetic acid, succinic acid, and lactate content, respectively, in conditioned media samples. Statistical difference is indicated in the charts with an asterisk (* for p≤0.05, ** for p≤0.01, *** for p≤0.001, and **** for p≤0.0001).

FIGS. 15A and 15B show the alkaline phosphatase (“ALP”) content in the growth media and the differentiation media, respectively. FIG. 15C shows the ALP content in the differentiation media. ALP is a protein commonly found in bone and is often used in determining osteogenic functionality of cells. Cells cultured in the static flask and both bioreactor configurations demonstrated ALP production. After 14 days of culture, it was shown that both bioreactor systems yielded cells that supported significantly higher ALP production than the static flask.

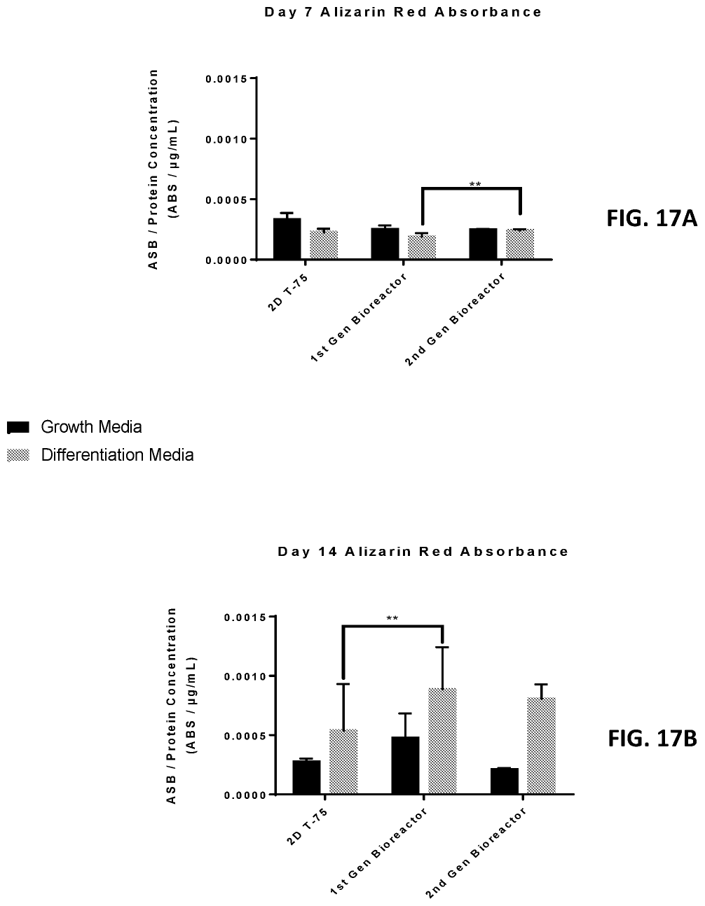

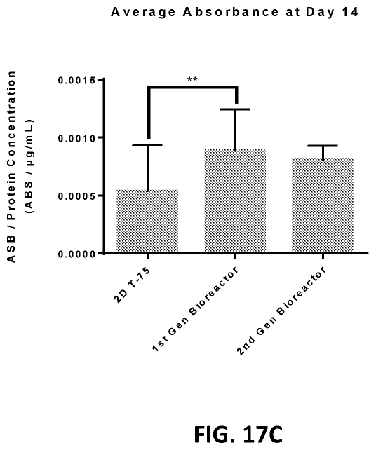

FIG. 16 is a microscopic image of cells stained with Alizarin Red. FIGS. 17A and 17B show the Alizarin Red absorbance at day 7 and day 14, respectively, and FIG. 17C shows the average absorbance at day 14. Cells cultured in growth media (FIGS. 16, A, C, and E) showed no stained mineral after 14 days for cells from each vessel, as expected. Cells from each vessel cultured with osteogenic differentiation media all exhibited stained mineral by day 14, indicated they maintained differentiation potential in all culture systems. Quantification of the destained mineral for each condition showed that the cells from the bioreactor systems had higher mineralization than cells than the static flask.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A bioreactor comprising:
a vessel comprising a wall with an interior surface defining a volume, and a fluid outlet;
a cell support insert removably disposed within the vessel;
a lid constructed to close the vessel;
a fluid inlet; and
a fluid distributor in fluid communication with and arranged to receive a fluid flow from the fluid inlet, the fluid distributor comprising a plurality of fluid delivery ports constructed to deliver fluid onto the cell support insert.

2. The bioreactor of claim 1, wherein the fluid inlet is on the lid.

3. The bioreactor of claim 1, wherein the fluid inlet is coupled with a pump supplying a fluid flow to the fluid inlet.

4. The bioreactor of claim 1, wherein the cell support insert comprises a disk.

5. The bioreactor of claim 1, wherein the cell support insert comprises a carrier configured to support microcarrier beads or culture substrates.

6. The bioreactor of claim 1, wherein the lid is coupled with the vessel via screw threading.

7. The bioreactor of claim 1, wherein the lid is coupled with the vessel via friction fit.

8. The bioreactor of claim 1, wherein the vessel has a cylindrical shape.

9. The bioreactor of claim 1 comprising a plurality of cell support inserts removably disposed within the vessel.

10. The bioreactor of claim 9, wherein the plurality of cell support inserts are stacked within the vessel.

11. The bioreactor of claim 1, wherein the lid comprises the fluid distributor.

12. The bioreactor of claim 1, wherein the fluid distributor forms a seal against the interior surface of the vessel.

13. The bioreactor of claim 1, wherein the fluid distributor comprises a branched fluid flow channel comprising an inlet end and terminating at the plurality of fluid delivery ports.

14. The bioreactor of claim 1, wherein the fluid distributor comprises a top wall comprising an opening and an opposing bottom wall comprising a plurality of openings comprising the plurality of fluid delivery ports.

15. The bioreactor of claim 14, wherein the opening in the top wall defines the fluid inlet.

16. The bioreactor of claim 1, wherein the plurality of fluid delivery ports comprises 3 or more ports.

17. The bioreactor of claim 1, wherein the cell support insert defines an area perpendicular to a longitudinal axis of the vessel, and wherein the plurality of fluid delivery ports comprises 1 port per every 10 cm² or less of the area.

18. The bioreactor of claim 1, wherein the fluid distributor is spaced apart from the cell support insert by a distance of 8 mm or greater.

19. The bioreactor of claim 1, wherein the fluid distributor is spaced apart from the cell support insert by a distance of 30 mm or less.

20. The bioreactor of claim 1, wherein the fluid distributor is arranged to apply a shear force of 0.15 Pa or less to cells when fluid is circulated at a rate of 25 mL/min to 125 mL/min.

21. A bioreactor system comprising:
the bioreactor of claim 1;
a pump operatively coupled with the fluid inlet; and
a volume of cell culture media,
wherein the bioreactor system is a closed system.

22. A method of culturing cells, the method comprising:
applying cell culture media and prepared cells to the vessel of the bioreactor of claim 1; and
circulating the cell culture media from the fluid outlet to the fluid inlet.

23. The method of claim 22, wherein the cell culture media is circulated at a rate of 25 mL/min or greater.

24. The method of claim 22, wherein the circulating of the cell culture media applies a shear force of 0.15 Pa or less.
